(19) United States
(12) Patent Application Publication
(10) Pub. No.: US 2013/0085472 A1
(43) Pub. Date: Apr. 4, 2013

(54) MICRONEEDLE NASAL DELIVERY DEVICE

(71) Applicant: Christopher Shaari, Demarest, NJ (US)

(72) Inventor: Christopher Shaari, Demarest, NJ (US)

(73) Assignee: Toxcure, LLC, Demarest, NJ (US)

(21) Appl. No.: 13/583,446

(22) PCT Filed: Mar. 9, 2011
(86) PCT No.: PCT/US11/27745

§ 371 Date: Dec. 19, 2012

Related U.S. Application Data
(60) Provisional application No. 61/312,078, filed on Mar. 9, 2010.

(51) Int. Cl. A61M 37/00; A61M 5/00; A61M 5/32; A61M 25/10

(52) U.S. Cl. CPC A61M 37/0015; A61M 25/10; A61M 5/00; A61M 5/3286
USPC 604/506; 604/173; 604/96.01; 604/272; 604/257; 604/239

(57) ABSTRACT

The invention is directed to a nasal delivery device comprising one or more microneedles, and to methods of nasally administering a composition with a nasal delivery device comprising one or more microneedles. In certain embodiments, the nasal delivery device comprises a substrate for administration of a composition to the nasal and/or sinus mucosa, wherein the substrate is non-absorbent and comprises one or more microneedles. In some embodiments, the nasal delivery device comprises a reservoir comprising one or more therapeutic agents, wherein the reservoir is in fluid communication with one or more microneedles.

### RELATED APPLICATION

This application claims the benefit of priority of U.S. Provisional Application Ser. No. 61/312,078, filed Mar. 9, 2010. The foregoing application is incorporated herein by reference in its entirety.

### FIELD OF THE INVENTION

The invention relates generally to nasal delivery devices and to methods of delivering a composition to a desired location within the nasal cavity of a patient. More particularly, the invention is directed to a nasal delivery device comprising one or more microneedles, and to methods of nasally administering a composition with a nasal delivery device comprising one or more microneedles.

### BACKGROUND OF THE INVENTION

Intranasal drug delivery has been recognized as a useful and reliable alternative to oral and parenteral drug delivery routes. Intranasal administration of medication for the symptomatic relief and prevention or treatment of topical nasal conditions has been widely used for a long period of time. However, recently, the nasal mucosa has emerged as a therapeutically viable route for systemic drug delivery. In general, among the primary targets for intranasal administration are pharmacologically active compounds with poor stability in gastrointestinal fluids, poor intestinal absorption and/or extensive hepatic first-pass elimination, such as peptides, proteins and polar drugs. The foregoing is reviewed, for example, in Pires, A et al. J Pharm Pharmaceut Sci 12(3) 288-311, 2009.

Transnasal delivery can be used for conditions related to the nose and sinuses (sinonasal) or for conditions unrelated to the nose and sinus. Transnasal delivery traditionally involves either topical application to the nasal lining or injection into the nasal lining.

Nasal administration of a compound poses a number of unique challenges in comparison to other modes of administration, such as, for example, transdermal application of a compound. Whereas dermal application is clearly visible, nasal application is not. The target of the nasal delivery is the sinonasal mucosa that includes but is not limited to the turbinate, septal and sinus lining. A device for nasal administration of a compound must be capable of reaching these regions that sometimes requires a lighted scope (endoscope or lighted speculum) to reach. In addition, pain and pressure nerve endings are abundant throughout the sinonasal cavity, rendering it highly sensitive. Furthermore, it is a highly vascular area, and to prevent bleeding, care must be taken to not puncture blood vessels.

Moreover, the nasal lining is covered in a mucous blanket that protects the underlying mucosa. This blanket serves as a physical barrier to the passage of medicine when delivered topically. Digestive enzymes also line the mucosa that further limit passage of protein molecules. This is reviewed in Indian J Pharmacol|June 2004|Vol 36|Issue 3|140-147: The nasal epithelium is covered by a mucus layer that is renewed every 10 to 15 min. The pH of mucosal secretion ranges from 5.5 to 6.5 in adults and from 5.0 to 6.5 in children. The mucus layer entraps particles, which are then cleared from the nasal cavity by the cilia. The rate of mucus flow through the nose is approximately 5 to 6 mm/min resulting in particle clearance within the nose every 20 min. The nasal cavity also houses numerous enzymes. In humans, cytochrome P450 enzyme isoforms that have been identified are CYP1A, CYP2A and CYP2E. Other enzymes detected in the human nose include carboxylesterases and glutathione S-transferases.

Topical application is most commonly used to treat conditions related to the nose and sinus. Such therapy includes topical nasal steroids such as FLONASE®, NASONEX®, NASACORT AQ®, RHINOCORT®; topical nasal antihistamines such as ASTELIN® or ASTEPRO®; topical nasal anticholinergics such as ATROVENT®; topical nasal vasoconstrictors such as AFRIN®, NEOSNEPHRINE®; topical nasal antibiotics or antifungals such as gentamicin or amphotericin; and topical nasal mucolytics such as mucomyst.

Topical application of medication for non sinonasal conditions includes use of midazoloam spray for seizure management, fentanyl for cancer pain, and naloxone for heroin overdoses.

Current Methods and devices that provide topical nasal delivery include aerosolization (MUCOSAL ATOMIZATION DEVICE®, OPTINOSE®), nebulization (SINUNEB®), and placement of merocel or dissolvable device soaked with product. Ampules can be discharged as well. Recently, Acclarent, Inc. introduced RELIEVA STRATUS®, a drug eluting stent that is surgically placed in the sinus cavity and allows gradual application of drug to the ethmoid sinus.

A system for delivering toxin and toxin fragments to a patient's nasal cavity was recently described that utilizes energy to porate target cells (US2008/0021369 A1).

Topical delivery is limited by the presence of a mucous blanket that lines the surface of the nasal or sinonasal mucosa and provides a physical barrier to the absorption of molecules. Enzymes exist in the nasal mucus blanket that potentially breakdown protein molecules. Tight junctions also limit transmucosal passage of molecules across the mucosa. Also, topical delivery is not typically directed toward one particular region of the nose, thereby exposing regions of the sinonasal cavity such as the olfactory region to medication that may lead to complications. Medication can also leak into the back of the nose and be swallowed, thereby exposing the patient to systemic effects.

Injection therapy of the nose is a less common modality because it is an invasive therapy that requires specialized training and carries unique complications such as bleeding, or blindness secondary to embolization or induced vasospasm. Only a few classes of medication are typically injected into the nose and include nasal steroids and local anesthetics. Although injection therapy may be efficacious, it carries more risks than topical administration.

Nasal steroid injections are usually reserved for patients with allergic rhinitis, nonallergic rhinitis and nasal polyposis. Steroids have been injected into the olfactory cleft of patients with olfactory loss as well (Chem Senses. 2005 January; 30 Suppl 1:i212-3).

Visual loss from turbinate injections is a rare but potentially devastating complication, and has resulted in the significant reduction of nasal steroid injections. The visual symptoms are transient for most reported cases, but permanent loss of vision has been reported. The estimated incidence rate of visual loss after injection into the inferior turbinates has been estimated at 0.006% (Otolaryngol Head Neck Surg 2003; 128:280-1).

The mechanism is thought to be due to an intravascular arterial injection of steroid in the nose followed by retrograde flow of small particles into the ophthalmic system then into the eye. The injection can embolize the small diameter vessels of the retinal artery and produces retinal edema. Another theory is that larger vessels can go into vasospasm, resulting in blindness as well.

Injections leading to blindness have included methylprednisolone, which is composed of particles 99% of which are 20 microns or less and 75% of which are 10 microns or less (Arch Ophthalmol 97:79-80, 1979). A report of blindness has occurred when triamcinolone (Kenalog) has been used as well. Triamcinolone is composed of particles in which 90% of the particles are 10 microns or less.

Methods to avoid blindness included the application of topical decongestants and anesthetizing agents to the nasal mucosa before injecting; injecting slowly, under least pressure and in small quantities; aspiration to confirm lack of intraarterial location; avoiding solutions with large particles that are more likely to occlude arteries (small particles appear to be better tolerated); and moving the needle to avoid a large bolus of injection.

Local anesthetics are routinely injected in the office or in the operating room for those undergoing surgery, such as correction of a deviated septum, turbinate abnormalities or nasal or sinonasal conditions.

### SUMMARY OF THE INVENTION

The present invention provides a more effective way of delivering medications to the sinonasal cavity, can be performed by any healthcare provider, and is associated with minimal side effects and potentially delivers higher therapeutic efficacy than current topical or injection techniques alone. The instant device can be used to treat sinonasal and non-sinonasal conditions.

In certain embodiments, the invention provides a nasal delivery device comprising a substrate suitable for administration of a composition to the nasal mucosa, wherein the substrate comprises at least one microneedle. In other embodiments, the substrate comprises a plurality of microneedles. Examples of substrates include cotton, sponges, inflatable balloons, probes, rollers, and polymeric silicone elastomers (e.g., SILASTIC® sheets).

In certain embodiments, the at least one microneedle is 150 micrometers or less in length. In some embodiments, the microneedle is hollow. In other embodiments, the microneedle is porous.

In some embodiments, the nasal delivery device comprises a reservoir in fluid communication with the at least one microneedle. In some embodiments, the device comprises a shaft. In certain embodiments, the device comprises a shaft, wherein the shaft comprises a hollow central tubing in fluid communication with the reservoir and the at least one microneedle. In a particular embodiment, the central tubing is fenestrated. In certain embodiments, the shaft and/or the reservoir comprise an injection port. In some embodiments, the injection port is suitable for attachment of a syringe.

In some embodiments, the device comprises a shaft, wherein the shaft is calibrated, bendable, and/or angled. In yet other embodiments, the device comprises a shaft with a raised barrier. In certain embodiments, the raised barrier is at most 2.5 cm from the end of the device comprising the at least one microneedle. In certain embodiments, the raised barrier is at most 1.5 cm from the end of the device comprising the at least one microneedle.

In some embodiments, the substrate is concaved, and the plurality of microneedles are attached or integrated on one side of the concave surface. In other embodiments, the substrate is convex, and the plurality of microneedles are attached or integrated on one side of the convex surface. In yet other embodiments, the substrate is flat, and the plurality of microneedles are attached or integrated on one side of the flat surface. In some embodiments, the plurality of microneedles are attached or integrated on both sides of the surface.

In some embodiments, the nasal delivery device comprises an absorbent substrate that is a polyvinyl alcohol sponge. In some embodiments, the substrate is a polyvinyl alcohol sponge, wherein the plurality of microneedles are attached or integrated onto one side of the sponge. In other embodiments, the plurality of microneedles are attached or integrated onto both sides of the sponge. In yet other embodiments, the polyvinyl alcohol sponge comprises a soft pliable plastic sheet, wherein the microneedles are attached or integrated onto the sheet.

One or more compositions can be administered to the nasal and/or sinus mucosa with a nasal delivery device as described herein. In some embodiments, the nasal delivery device of comprises one or more compositions at predetermined dosages. Examples of compositions that can be administered include adreno corticosteroids, antibiotics, antimigraine drugs, antiviral drugs, cardiovascular drugs, central nervous system drugs, autonomic nervous system drugs, diagnostic drugs, histamine, antihistamines, narcotics, sex hormones, inorganic compounds, vitamins, peptides, polypeptides, and proteins. In certain embodiments, the composition is coated onto at least a portion of the device.

In other embodiments, the invention relates to a method for the nasal administration of a composition, comprising (a) contacting a composition with a nasal delivery device, wherein the nasal delivery device comprises a substrate suitable for administration of the composition to the nasal or sinus mucosal surface, wherein the substrate comprises one or more microneedles, and wherein the composition coats at least a portion of the one or microneedles of the device; and (b) contacting the nasal delivery device of (a) with a nasal or sinus mucosal surface such that the one or microneedles penetrate at least a portion of the mucosal surface, wherein the composition is nasally administered. In some embodiments, the nasal delivery device of (a) contacts the composition by dipping the device in a solution or gel comprising the composition. In certain embodiments, the method further comprises cleaning the nasal or sinus mucosal surface with an absorbent substrate prior to contacting the surface with the one or more microneedles. In other embodiments, the method further comprises cleaning the nasal or sinus mucosal surface with an absorbent substrate after contacting the surface with the one or more microneedles. In some embodiments, the method further comprises cleaning the nasal or sinus mucosal surface with a non-absorbent substrate (e.g., a plastic wipe, silicone, SILASTIC®) prior to contacting the surface with the one or more microneedles. In other embodiments, the method further comprises cleaning the nasal or sinus mucosal surface with a non-absorbent substrate after contacting the surface with the one or more microneedles. The absorbent or non-absorbent substrate can be curved and/or straight. Examples of nasal or sinus mucosal surfaces include the nasal vestibule, turbinates, septum, lateral wall, middle meatus, superior meatus, inferior meatus, and the olfactory mucosa and epithelium.

In some embodiments, the substrate of the nasal delivery device of (a) comprises a shaft with a raised barrier. In certain embodiments, the raised barrier is situated at most 2.5 cm from the end of the device comprising the one or more microneedles. In further embodiments, the nasal mucosal surface is squamous lining. In other embodiments, the raised barrier is situated at most 1.5 cm from the end of the device comprising the one or more microneedles. In further embodiments, the nasal mucosal surface is pseudostratified columnar epithelium, olfactory mucosa, and/or neuroepithelium.

In some embodiments, the substrate of the nasal delivery device of (a) is cotton, wherein the cotton is at one end of a shaft, and the one or more microneedles are attached or integrated in the cotton, and wherein the shaft comprises a fenestrated central tubing in fluid communication with the reservoir and the one or more microneedles.

In certain embodiments, the substrate of the nasal delivery device is cotton, wherein the device comprises cotton at one end of a shaft, and the one or more microneedles are attached or integrated in the cotton. In other embodiments, the substrate is non-absorbent, wherein the non-absorbent substrate is attached to one end of a shaft, and the one or more microneedles are attached or integrated in the non-absorbent substrate. In certain embodiments, the non-absorbent substrate is a roller. In some embodiments, the roller is cylindrical and rotates freely relative to the shaft. In other embodiments, the roller is partially cylindrical. In certain embodiments, the shaft comprises an injection port. In further embodiments, the injection port is suitable for attachment of a syringe. In some embodiments, the shaft comprises a hollow center tubing.

In some embodiments, the nasal delivery device further comprises an absorbent substrate at the opposite end of the shaft. In certain embodiments, the absorbent substrate is a sponge. In further embodiments, the absorbent substrate is a polyvinyl alcohol sponge. In other embodiments, the nasal delivery device further comprises a non-absorbent substrate at the opposite end of the shaft. In certain embodiments, the non-absorbent substrate is plastic.

In some embodiments, the invention relates to a method for the systemic delivery of a composition, comprising (a) contacting a composition with a nasal delivery device, wherein the nasal delivery device comprises a substrate suitable for administration of the composition to the nasal or sinus mucosal surface, wherein the substrate comprises one or more microneedles, and wherein the composition coats at least a portion of the one or microneedles of the device; and (b) contacting the nasal delivery device of (a) with a nasal or sinus mucosal surface, such that the one or microneedles penetrate at least a portion of the mucosal surface, wherein the composition is systemically delivered. In certain embodiments, the mucosal surface is the olfactory mucosa. In further embodiments, the composition is delivered to the central nervous system. In yet other embodiments, the nasal delivery device of (a) contacts the composition by dipping the device in a solution or gel comprising the composition.

In some embodiments, the invention relates to a method for the CNS delivery of a composition, comprising (a) contacting a composition with a nasal delivery device, wherein the nasal delivery device comprises a substrate suitable for administration of the composition to the nasal or sinus mucosal surface, wherein the substrate comprises one or more microneedles, and wherein the composition coats at least a portion of the one or microneedles of the device; and (b) contacting the nasal delivery device of (a) with an olfactory mucosal surface, such that the one or microneedles penetrate at least a portion of the olfactory mucosal surface, wherein the composition is delivered to the CNS. In yet other embodiments, the nasal delivery device of (a) contacts the composition by dipping the device in a solution or gel comprising the composition.

In some embodiments, the method relates to a method for the nasal administration of a composition, comprising (a) applying a solution or gel comprising a composition to a nasal or sinus mucosal surface; (b) contacting the nasal or sinus mucosal surface with a nasal delivery device comprising a substrate, wherein the substrate is a roller and comprises one or more microneedles; and (c) rolling the roller of the microneedle device such that the one or more microneedles penetrate at least a portion of the nasal or sinus mucosal surface, wherein the composition is nasally administered.

### BRIEF DESCRIPTION OF THE DRAWINGS

FIG. 1 (A) depicts a cotton-tipped applicator to which are attached microneedles on all sides of the cotton tip, the cotton tip is situated at one end of the shaft, and a sponge is situated at the other end of the shaft; (B) depicts a cross-sectional view of a cotton tip comprising microneedles around the outside of the cotton; (C) depicts an applicator with a pointed tip to which are attached microneedles on one side of the tip, the pointed tip is situated at one end of the shaft, and a sponge is situated at the other end of the shaft; (D) depicts a cross-sectional view of a pointed tip comprising microneedles on one side of the tip; and (E1) depicts a curved pointed tip to which are attached microneedles on the concave side of the tip, the pointed tip is situated at one end of the shaft, and a sponge is situated at the other end of the shaft, (E2) depicts the microneedles on the convex side of the tip, and (E3) depicts the microneedles on both sides of the curved tip.

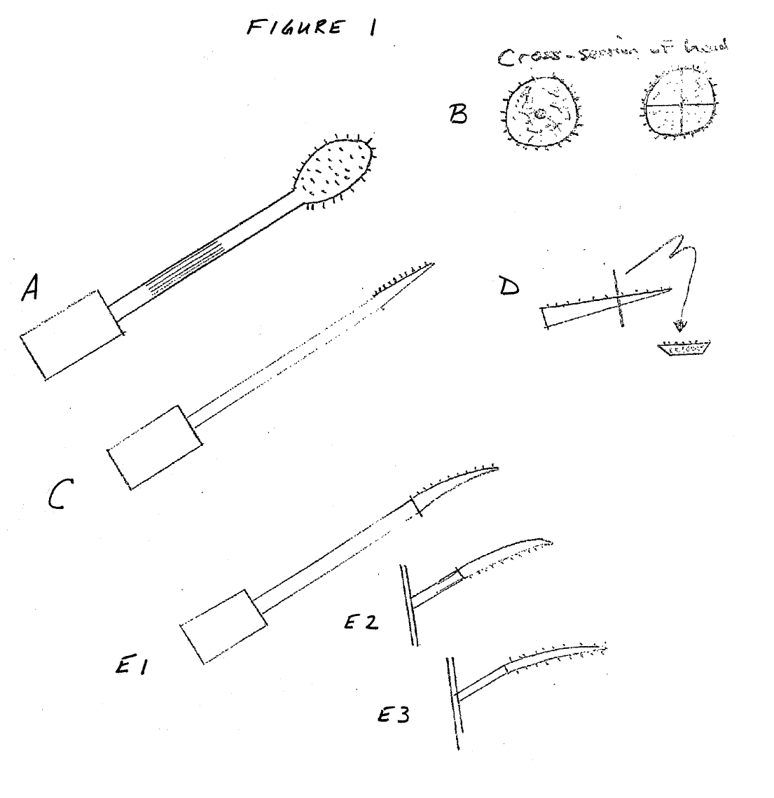

FIG. 2 (A1) depicts a cotton-tipped applicator to which are attached microneedles on all sides of the cotton tip, the cotton tip is situated at one end of the shaft, and a sponge is situated at the other end of the shaft, and the shaft comprises a reservoir and central tubing in fluid communication with the microneedles, (A2) depicts a cross-sectional view of the shaft with central tubing, (A3) depicts fenestrated central tubing, and (A4) depicts the reservoir and fenestrated central tubing in fluid communication with the microneedles, whereby the fluid is ejected from the central tubing into the cotton tip comprising microneedles.

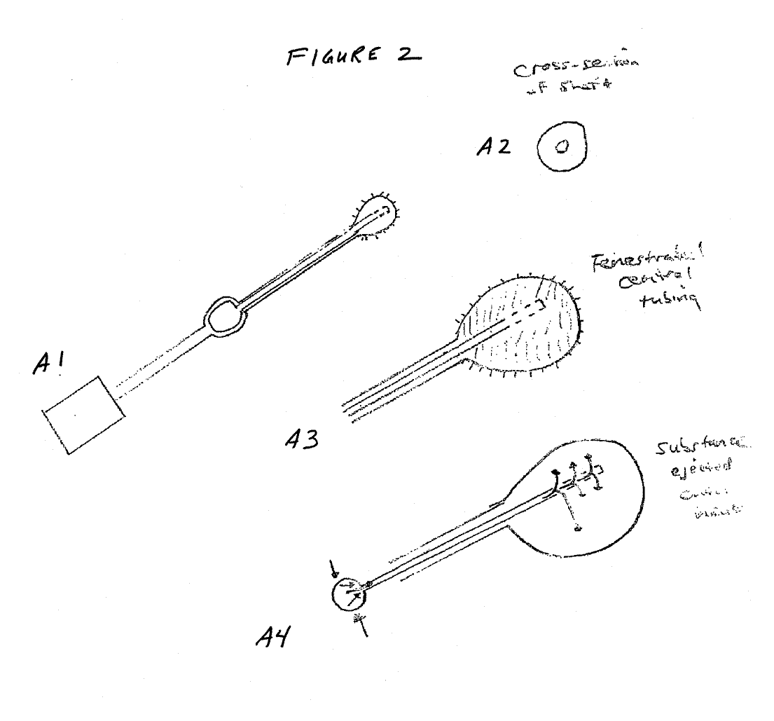

FIG. 3(A) depicts (1) a microneedle nasal delivery device according to the invention with an angled shaft and (2) with a bendable shaft; (B) depicts a microneedle nasal delivery device according to the invention with a calibrated shaft; and (C) depicts microneedle nasal delivery devices according to the invention with a barrier.

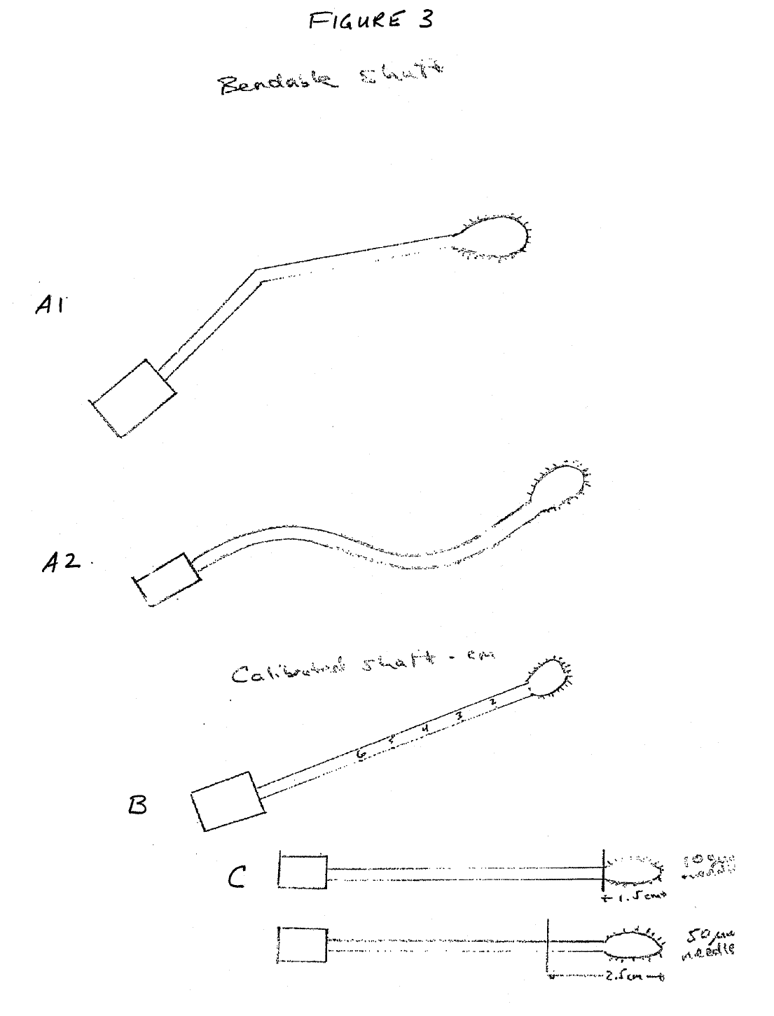

### DETAILED DESCRIPTION OF THE INVENTION

The present invention provides a novel device for the nasal administration of a composition. The invention described herein relates to a nasal delivery device comprising one or more microneedles. The one or more microneedles are typically attached to an outer surface of the device and are suitable for the administration of a composition to the nasal mucosa. The invention described herein also provides novel methods of administering a composition, such as a biologically active compound, locally, systemically, and/or to the central nervous system (CNS).

The nasal delivery device of the invention comprises a substrate to which is attached at least one microneedle. Typically, a nasal delivery device of the invention will comprise a plurality of microneedles. The substrate comprising the one or more microneedles may be constructed from any material suitable for administration to the nasal cavity, including organics, polymers, metals, ceramics, semiconductors, and composites. Examples of suitable substrates include inflatable balloons, probes, rollers, and absorbent materials such as cotton and sponges (e.g., polyvinyl alcohol sponges such as MEROCEL® sponges).

The skin has been the target of most microneedle technology. The eye has also been used targeted, in the form of intrascleral or intracorneal routes (Investigative Ophthalmology and Visual Science. 2007; 48:4038-4043). With microneedle technology, multiple tiny pores are typically created in the most superficial layer of the skin (stratum corneum) using an array of microneedles. The needles can penetrate to any predetermined length but usually penetrate the layer that is superficial to the nerve endings and blood vessels, thereby creating a painless and bloodless injection technique. Therapeutic level of medication is reached, which is sometimes more effective than injection alone.

The term “microneedle” typically refers to a needle having a diameter at most about 100 μm, preferably about 10 μm or less and a length at most about 1 mm. As used herein, the term “microneedle” refers to any needle-like structure having a height above the substrate surface from which they protrude of about 500 micrometers or less. Preferably, the microneedle is less than 200 μm. In a preferred embodiment, the height of the microneedle may be about 150 μm, about 125 μm, about 100 μm, about 75 μm, about 70 μm, about 65 μm, about 60 μm, about 55 μm, about 50 μm, about 45 μm, about 40 μm, about 35 μm, about 30 μm, about 25 μm, about 20 μm, about 15 μm, about 10 μm or less. The length of the needle is selected for the particular application, accounting for both an inserted and uninserted portion. An array of microneedles can include a mixture of microneedles having, for example, various lengths, outer diameters, inner diameters, cross-sectional shapes, and spacings between the microneedles.

It is desirable that the height of the microneedle of the present invention is sufficient to pass through the nasal and/or sinus mucosa. In preferred embodiments, the height of the microneedle is sufficient to deliver a composition within the nasal and/or sinus mucosa or epithelium. It is also desirable that the height of the microneedle is not sufficiently large to stimulate nerves in deeper tissue and cause pain or to rupture blood vessels and cause bleeding when inserted at a delivery site. In preferred embodiments, the height of the microneedle is less than 200 microns (micrometers), e.g., less than 100 microns, thereby avoiding the pain fibers and blood vessels that are located beneath the nasal and sinus epithelium. For example, the thickness of the nasal and/or sinus mucosal lining is typically 50-150 microns. Accordingly, in certain embodiments, to avoid pain and/or bleeding upon nasal administration of a composition, the microneedle should penetrate no more than 150-200 microns.

The microneedles applicable to this invention include ones that are hollow. The term “hollow” means having one or more lumen(s) running through the interior of the microneedle, wherein fluid and/or solid materials can pass through the lumen(s). These hollow microneedles can preferably have an aperture connected to the lumen of the micro-needle. The term “aperture” means an opening in the outer surface of the microneedle which is sufficiently large to allow passage of fluid and/or solid materials out of the micro-needles. The aperture can be at the tip of the microneedles or located at other places in the microneedle outer surface. In other embodiments, the microneedles can be solid or capable of being ruptured.

The selection of the microneedles to serve for the nasal delivery of a composition can vary widely within the scope of the invention. Microneedles may be manufactured from a variety of materials. Material selection may be based on a variety of factors including, for example, the ability of the material to accurately reproduce a desired pattern, the strength and toughness of the material when formed into the microneedles, the compatibility of the material with, for example, human or animal skin, and the compatibility of the materials with any fluids that will be expected to contact the microneedle devices. Microneedles may be constructed from, for example, glassy materials, metals, ceramics, semiconductors, organics, polymers, including biodegradable polymers, composites, and combinations of such materials. Suitable examples of materials of construction include pharmaceutical grade stainless steel, gold, titanium, nickel, iron, gold, tin, chromium, copper, alloys of these or other metals, silicon, silicon dioxide, and polymers. Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polymethacrylic acid, ethylenevinyl acetate, polytetrafluorethylene (TEFLON®), and polyesters. Among polymeric materials it may be preferred that the microneedles be manufactured of thermoplastice materials. Such suitable polymeric materials for the microneedles of the present invention may include, but are not limited to: acrylonitrile-butadiene-styrenes, polyphenyl sulfides, polycarbonates, polypropylenes, acetals, acrylics, polyetherimides, polybutylene terephthalates, polyethylene terephthalates, etc. Polymeric microneedles may be manufactured from a single polymer or a mixture/blend of two or more polymers.

Generally, microneedles should have the mechanical strength to remain intact while being inserted into the nasal and/or sinus mucosa and while being removed from the mucosa. Also, in some embodiments, it may be desirable to leave the microneedle device attached to the mucosal surface to provide continuous delivery of a composition. For such continuous delivery, it is desired that the microneedles remain intact while remaining in place for up to a number of days. Another approach is for some or all of the microneedle to detach and remain in the mucosa, for example if a biodegradable material is used.

The microneedle structure of the microneedle devices of the present invention can be porous, solid, or hollow. As used herein, the term “porous” means having pores or voids throughout at least a portion of the microneedle structure, sufficiently large and sufficiently interconnected to permit passage of fluid and/or solid materials through the microneedle. As used herein, the term “hollow” means having one or more substantially annular bores or channels through the interior of the microneedle structure, having a diameter sufficiently large to permit passage of fluid and/or solid materials through the microneedle. The annular bores may extend throughout all or a portion of the needle in the direction of the tip to the base, extending parallel to the direction of the needle or branching or exiting at a side of the needle, as appropriate. A solid or porous microneedle can be hollow. One of ordinary skill in the art can select the appropriate porosity and/or bore features required for specific applications.

In some embodiments, the movement of a fluid toward or away from the microneedles may be accomplished by a capillary wicking action. In such instances, coatings may be provided, for example, hydrophilic coatings, that enhance the capillary wicking action.

The microneedles may have straight or tapered shafts. Microneedles may be formed with shafts that have a circular cross-section in the perpendicular, or the cross-section can be non-circular. The cross-sectional dimensions may be between 10 nanometers and 1 millimeter, e.g., between 1 micrometer and 200 micrometers, between 10 micrometers and 100 micrometers. Microneedles can be oriented perpendicular or at an angle to the substrate. The outer diameter is typically between about 10 μm and about 100 μm, and the inner diameter is typically between about 3 μm and about 80 μm.

The microneedles can be formed with shafts that have a circular cross-section in the perpendicular, or the cross-section can be non-circular. For example, the cross-section of the microneedle can be polygonal (e.g., star-shaped, square, triangular), oblong, or another shape. The shaft can have one or more bores.

The microneedles can be oriented perpendicular or at an angle to the substrate. In some embodiments, the microneedles are oriented perpendicular to the substrate so that a larger density of microneedles per unit area of substrate can be provided.

In some embodiments, the substrate and/or microneedles, as well as other components, are formed from flexible materials to allow the device to fit the contours of the various regions of the nasal cavity, including the nasal and/or sinus mucosa, to which the device is applied.

The microneedles may be arranged in a variety of arrays, including on sheets, rollers, or sheaths made from any number of suitable materials, such as polymeric silicone elastomers (e.g., SILASTIC® materials). An array of microneedles can include a mixture of microneedle orientations, heights, or other parameters. In some embodiments, a plurality of microneedles are attached or integrated on both sides of the substrate surface. In certain embodiments, a plurality of microneedles are attached or integrated on a SILASTIC® sheet or other material that is wrapped around the substrate surface. The substrate surface may be any shape, including concave, convex, and/or flat.

A microneedle device of the invention may include a reservoir in communication with the microneedles attached to a substrate for nasal delivery a composition. The reservoir can be attached to the substrate by any suitable means. In one embodiment, the reservoir is attached to the back of the substrate (opposite the microneedles) around the periphery, using an adhesive agent (e.g., glue). A gasket may also be used to facilitate formation of a fluid-tight seal.

The reservoir may be a hollow vessel, a porous matrix, or a solid form including drug that is transported therefrom. The reservoir can be formed from a variety of materials that are compatible with the drug or biological fluid contained therein. Suitable materials include natural and synthetic polymers, metals, ceramics, semiconductors, organics, and composites.

In a certain embodiment, the reservoir should be in direct contact with the microneedles and have holes through which drug could exit the reservoir and flow into the interior of hollow or porous microneedles. In another embodiment, the reservoir has holes which permit the drug to transport out of the reservoir and onto the nasal and/or sinus mucosal surface. From there, drug is transported into the mucosa, either through hollow or porous microneedles, along the sides of solid microneedles, or through pathways created by microneedles in the mucosa.

If hollow microneedles are used, the hollow center may be in fluid communication with the fluid reservoir. The fluid reservoir may contain one or more compounds and/or other agents. A microneedle device may also include a pump and/or microprocessor.

The microneedle device can include one or a plurality of chambers for storing materials to be delivered. In an embodiment having multiple chambers, each can be in fluid connection with all or a portion of the microneedles of the device array. In one embodiment, at least two chambers are used to separately contain drug (e.g., a lyophilized drug, such as a vaccine) and an administration vehicle (e.g., saline) in order to prevent or minimize degradation during storage. Immediately before use, the contents of the chambers are mixed. Mixing can be triggered by any means, including, for example, mechanical disruption (e.g., puncturing or breaking), changing the porosity, or electrochemical degradation of the walls or membranes separating the chambers. In another embodiment, a single device is used to deliver different drugs, which are stored separately in different chambers.

In certain embodiments, the rate of delivery of each drug can be independently controlled. The rate of drug delivery can be controlled by varying a number of design factors, including the outer diameter of the microneedle, the number and size of pores or channels in each microneedle, the number of microneedles in an array, the magnitude and frequency of application of the force driving the drug through the microneedle and/or the holes created by the microneedles. For example, devices designed to deliver drug at different rates might have more microneedles for more rapid delivery and fewer microneedles for less rapid delivery. As another example, a device designed to deliver drug at a variable rate could vary the driving force (e.g., pressure gradient controlled by a pump) for transport according to a schedule which was pre-programmed or controlled by, for example, the user or his doctor. The devices can be affixed to the nasal and/or sinus mucosa to deliver drugs continuously or intermittently, for durations ranging from a few seconds to several hours or days.

One or more microneedles may be attached to the substrate on the device in any suitable manner. For example, a sheet or sheath of microneedles may be overlaid over a cotton tip on a cotton-tipped applicator. In some embodiments, the sheet or sheath of microneedles are overlaid over a non-absorbent tip or applicator (e.g., a plastic tip or applicator). In some embodiments, the sheath is disposable and may be placed over the cotton tip immediately prior to usage. In another embodiment, the substrate is an inflatable balloon lined or coated with cotton to which a plurality of microneedles are attached.

In yet other embodiments, the substrate comprising the one or more microneedles is configured on a shaft or other suitable means for substrate attachment and device manipulation (e.g., insertion into the nasal cavity). The shaft may be any shape, including cylindrical and/or angled. In certain embodiments, the substrate is cotton and is attached to a shaft, e.g., a cotton-tipped applicator. In other embodiments, the substrate is a non-absorbent material (e.g., plastic) and is attached to a shaft. In some embodiments, the shaft comprises microneedles attached to a cotton tip at one end of the shaft, and the other end of the shaft comprises a sponge or other absorbent material.

In some embodiments, the shaft or other substrate attachment means comprises a raised barrier. The barrier may be useful for, among other things, regulating how far into the nasal cavity a device of the invention may be inserted. In these embodiments, the barrier may enable greater precision for drug administration, for example, by facilitating targeted administration of a therapeutic agent to a particular nasal and/or sinus mucosal surface. For example, in certain embodiments, a device of the invention may comprise a shaft with a raised barrier 1.5 cm from the tip of the inserted end of the device, thereby facilitating administration of a therapeutic agent to the squamous lining. In another particular embodiment, the device may comprise a shaft with a raised barrier 2.5 cm from the inserted end of the device, thereby facilitating administration of a therapeutic agent to the pseudostratified columnar epithelium.

The shaft or other substrate attachment means can be graduated, angled and/or bendable.

In some embodiments, the shaft comprises a reservoir such that the reservoir can be compressed, for example, by pressing the shaft between the fingers, such that one or more therapeutic agents are dispensed from the reservoir through one or more microneedles situated at one end of the shaft on an absorbent or non-absorbent substrate. In some embodiments, the substrate is a roller. In certain embodiments, the roller spins relative to the shaft, for example, where the tip of the shaft comprises a roller (e.g., one that is cylindrical in shape) that can spin freely relative to the shaft. In other embodiments, the tip of the shaft comprises a roller that is partially cylindrical and rolls when the shaft is spun between the fingers. In further embodiments, one or more therapeutic agents are dispensed from the reservoir upon compression of the reservoir in the shaft, and the microneedles are rolled in the nasal cavity of a patient in need of administration of the one or more therapeutic agents, thereby delivering the one or more therapeutic agents to the patient. In certain embodiments, the substrate comprising the one or more microneedles is non-absorbent, such as, for example, a plastic roller.

In some embodiments, the shaft comprises an injection port. In certain embodiments, the shaft comprises a reservoir that comprises an injection port. In some embodiments, the reservoir comprises a known amount (e.g., a fixed dose) of one or more therapeutic agents. In other embodiments, a syringe comprising a known amount (e.g., a fixed dose) of one or more therapeutic agents is attached to an injection port in the microneedle device. In another embodiment, the microneedle device of the invention comprises an empty reservoir that can be injected with a needle such that varying doses of one or more therapeutic agents can be loaded into the device prior to application in the nose. In further embodiments, the reservoir comprises a membrane.

Accordingly, known amounts of one or more therapeutic agents can be delivered to the nasal and/or sinus mucosa of a patient. For example, in some embodiments, the desired dose of a composition such as a therapeutic agent is delivered to the nasal and/or sinus mucosa of a patient by compressing a reservoir in the shaft of a microneedle device of the invention, wherein the reservoir comprises the desired dose of the composition and upon compression, disperses it through one or more microneedles situated at one end of the shaft on an absorbent or non-absorbent substrate, wherein the one or more microneedles penetrate at least a portion of the nasal and/or sinus mucosa, thereby delivering the desired dose of the one or more therapeutic agents to the nasal and/or sinus mucosa. In other embodiments, the desired dose is delivered via a syringe attached to an injection port in the microneedle device of the invention, wherein upon compression, the syringe disperses the desired dose of one or more therapeutic agents (e.g., by a hollow tube in the center of a shaft and connected to an injection port in the microneedle device of the invention) through one or more microneedles situated at one end of the shaft on an absorbent or non-absorbent substrate, wherein the one or more microneedles penetrate at least a portion of the nasal and/or sinus mucosa, thereby delivering the desired dose of the one or more therapeutic agents to the nasal and/or sinus mucosa.

In certain embodiments, the device comprises a cotton-tipped applicator to which are attached microneedles on all sides of the cotton tip, the cotton tip is situated at one end of the shaft, and a sponge is situated at the other end of the shaft. In other embodiments, the device comprises an applicator with a pointed tip to which are attached microneedles on one side of the tip, the pointed tip is situated at one end of the shaft, and optionally, a sponge is situated at the other end of the shaft. In certain embodiments, the tip is a curved pointed tip to which are attached microneedles on the concave side of the tip, the pointed tip is situated at one end of the shaft, and optionally, a sponge is situated at the other end of the shaft. In some embodiments, the microneedles are on the convex side of the tip. In other embodiments, the microneedles are on both sides of the curved tip.

In certain embodiments, the device comprises a cotton-tipped applicator to which are attached microneedles on all sides of the cotton tip, the cotton tip is situated at one end of the shaft, and a sponge is situated at the other end of the shaft, and the shaft comprises a reservoir and central tubing in fluid communication with the microneedles. In some embodiments, the central tubing is fenestrated and in fluid communication with the microneedles, whereby the fluid is ejected from the central tubing into the cotton tip comprising microneedles.

In some embodiments, the nasal delivery device is disposable. In certain embodiments, the nasal delivery device is used once. In other embodiments, the nasal delivery device is used more than once. In yet other embodiments, the nasal delivery device can be used as many times as is needed.

In some embodiments, a nasal delivery device of the invention is used in conjunction with an auxiliary device, such as, for example, a speculum. In embodiments where a speculum is used, the speculum may be a self-contained, lighted speculum. In certain embodiments, the speculum is disposable.

For the nasal delivery of more than one compound, the compounds may be contained within a fluid reservoir, the compounds may be coated onto the microneedle device, or, a combination thereof may be used, in which one or more compounds are contained within a reservoir and one or more compounds are coated onto the microneedle device. When more than one compound is delivered, the same or different concentrations and timings of delivery may be used for the various compounds.

Microneedle devices of the present invention may be sterilizable using standard methods. Microneedle devices may be designed for a single-use, with the device being disposed of after initial use. Alternatively, the devices of the present invention may be designed for repeated use.

Essentially any therapeutic or other bioactive agents can be delivered using these devices. Therapeutic agents can be proteins, enzymes, polysaccharides, polynucleotide molecules, and synthetic organic and inorganic compounds. Representative agents include anti-infectives, hormones, growth regulators, drugs regulating cardiac action or blood flow, and drugs for pain control. The therapeutic agent can be for local treatment or for regional or systemic therapy.

Examples of therapeutic agents that may be used with the device of the invention include adreno corticosteroids, antibiotics, antimigraine drugs, antiviral drugs, cardiovascular drugs, central nervous system drugs, autonomic nervous system drugs, diagnostic drugs, histamine, antihistamines, narcotics, sex hormones, inorganic compounds, vitamins, peptides, polypeptides, and proteins. In some embodiments, the therapeutic agent is selected from gentamicin, cephalosporin, penicillins, tyrothricin, dihydroergotamine, ergotamine tartrate, enviroxime, isosorbide dinitrate, propranolol, verapamil, hydralazine, nitroglycerin, clofilium tosylate, cocaine, lidocaine, diazepam, lorazepam, dopamine, dobutamine, ephedrine, epinephrine, phenylephrine, tramazoline, xylometazoline, methacholine, nicotine, atropine, prostaglandins, ipratropium, scopolamine, dye T-1824, phenolsulfonphthalein, potassium ferrocyanide, vital dyes, meclizine, disodium cromoglycate, buprenorphine, botulinum toxin, naloxone, estradiol, progesterone, norethindrone, testosterone, colloidal carbon, colloidal gold, inorganic salts, lead carbonate P, thorium B, folic acid, cyanocobalamin, amino acids, calcitonin, secretin, thyrotropin-releasing hormone, cerulean, leucine enkephalin, mekephamid pentagastrin, SS-6, substance P, kyotorphin, cholecystokinin, albumins, andrenocorticotropic hormone, gonadotropin releasing hormone, growth hormone, interferon, vaccines, horseradish peroxidase, insulin, glucagon, oxytocin, and vasopressin.

With solid needles, a therapeutic agent or other compound can be coated on the tips of the needles, or can penetrate around the needles into the nasal and/or sinus mucosal surface. Hollow needles can deliver a compound directly through the needles from a reservoir backing. Hollow needles can also withdraw fluid from the injected space to “sample” the fluid for analysis.

One or more therapeutic agents or other compound may be administered with a device of the invention to any area within the nasal and sinonasal cavity. Regions to which a compound may be applied with a device of the invention include the nasal vestibule, turbinates, septum, lateral wall, middle meatus, superior meatus, inferior meatus, and the mucosa, including the neuroepithelium, of the nasal and/or sinus surfaces. One or more compounds may be applied with a device of the invention to the sinonasal mucosa, including the olfactory mucosa and epithelium.

A compound may be applied, for example, to the inferior turbinate region, including anterior 1.5 cm of inferior turbinate, middle and posterior turbinate mucosa after 1.5 cm behind the nasal sill, as well as the superior, medial, lateral, or posterior sides. In another embodiment, a compound may be applied to the septum region, including the anterior, middle, or posterior septal mucosa and the septal swell body mucosa. In some embodiments, a compound may be applied to the lateral wall region, including surfaces adjacent to turbinate attachment.

Application of a compound with a device of the invention also includes application to the sinus mucosa, such as the mucosal surfaces that may be found in the middle meatus region, including the space and region lateral to the middle turbinate and containing the osteomeatal complex and unit. The middle meatus receives drainage from the paranasal sinuses (frontal, ethmoid, maxillary), and it is contemplated that the nasal delivery device of the invention can be used to administer one or more compounds to the sinus mucosa, including the mucosa of the middle meatus region. In other embodiments, the nasal delivery device of the invention can be used to administer one or more compounds to the mucosa of the superior (supreme) meatus, the region that receives drainage from the paranasal sinuses, in particular, the sphenoethmoid sinuses. In yet other embodiments, the nasal delivery device of the invention can be used to deliver a compound to the inferior meatus, the region that receives nasolacrimal duct.

In certain embodiments, a compound can be applied to the olfactory mucosa or epithelium with a device of the invention. This region includes the roof of the nasal cavity and extends down the septum and lateral nasal wall. The olfactory mucosa provide a unique gateway to the central nervous system (CNS), bypassing the traditional blood brain barrier.

The instant invention provides for local, systemic, and/or CNS delivery of one or more compounds. Local delivery refers to application of a compound to any region of the nasal and/or sinus mucosa and includes application of any number of therapeutic agents, including steroids, antibiotics, botulinum toxin, anesthetics and/or decongestants. Systemic delivery refers to the delivery of a compound to the systemic circulation. Systemic delivery may be carried out by administration of a compound with a device of the invention to any region of the nasal and/or sinus mucosa. Any number of therapeutic agents may be delivered systemically according to the invention, and include analgesics, cardiovascular drugs, hormones, insulin, antivirals, anti-migraine medications, steroids, and antihistimines. CNS delivery refers to the delivery of a compound into the CNS. CNS delivery may be carried out by administration of a compound with a device of the invention to any region of the nasal and/or sinus mucosa. In preferred embodiments, CNS delivery is carried out by administration of a compound to the olfactory mucosa with a device of the invention. Any number of therapeutic agents may be delivered to the CNS according to the invention, and include compounds for the treatment of Alzheimer's disease, epilepsy, brain tumors, trauma, pain control, dizziness and migraine or other headache conditions.

Microneedle delivery of a therapeutic agent to the nasal cavity with a device of the invention enables administration of medication to treat conditions of the sinonasal tract such as allergic rhinitis, non-allergic rhinitis, infectious rhinitis, acute sinusitis, chronic sinusitis, nasal polyposis, sinonasal polyposis, turbinate hypertrophy, septal deviation, nasal septal swell body enlargement, anosmia, hyposmia and others and headache conditions such as migraine, neuralgia and rhinogenic headache. In other embodiments, microneedle delivery of a therapeutic agent to the nasal cavity according to the invention enables administration of medication to treat conditions outside of the sinonasal tract that require systemic absorption such as diabetes, osteoporosis, allergies, arthritis, pain conditions, asthma, COPD, hypertension, migraine or other headache conditions and others. In yet other embodiments, nasal microneedle technology according to the invention can provide a portal of entry for immunotherapy.

Therapeutic agents that can be applied include nasal-related medications such as steroids, antihistamines, anticholinergics, leukotriene inhibitors, antibiotics, antifungals, and antivirals. Therapeutic agents that can be applied also include non-nasal related medications such as parathyroid hormone, central nervous system acting drugs, insulin, and anti-migraine medications.

Botulinum toxin application to the nasal cavity is a method of relieving the symptoms of allergic and non-allergic rhinitis, either through topical application or injection into the nose. Topical application has been via a spray or applied directly to a merocel sponge (Polyvinyl alcohol, MEROCEL®) which is left in place for several minutes. Injection has typically been to the turbinates within the nasal cavity. Both techniques have been successful at relieving associated symptoms of rhinitis. It is considered a safe and effective method of providing long term relief after one application. Limitations of the topical technique, however, include inefficient transport across the nasal mucosal barrier and non-selective application to the nasal cavity. Likewise, injection carries the risk of bleeding and pain at the injection site, with risk of injecting botulinum toxin directly into the vasculature.

Examples of local conditions that may be treated by nasal administration of a composition with a device as described herein include allergic rhinitis, including seasonal, perennial, episodic, and occupational rhinitis; and non-allergic rhinitis, including both acute and chronic non-allergic rhinitis, NARES syndrome (non-allergic rhinitis with eosinophilia syndrome), perennial non-allergic rhinitis (vasomotor rhinitis), and other rhinitis syndromes such as ciliary dyskinesia syndrome, atrophic rhinitis, hormonally induced (e.g., hypothyroidism, pregnancy, oral contraceptives, and menstrual cycle), exercise, drug-induced (e.g., rhinitis medicamentosa, oral contraceptives, anti-hypertensive therapy, aspirin, and nonsteroidal anti-inflammatory drugs), reflex-induced (e.g., gustatory rhinitis, chemical or irritant-induced, posture reflexes, nasal cycle, and emotional factors), and occupational. Examples of other local conditions that may be treated include conditions that may mimic symptoms of rhinitis, including structural/mechanical factors (e.g., deviated septum/septal wall anomalies, hypertrophic turbinates, adenoidal hypertrophy, benign and malignant nasal tumors, and choanal atresia) and inflammatory/immunologic (e.g., Wegener's granulomatosis, sarcoidosis, midline granuloma, systemic lupus erythematosus, Sjogren's syndrome, and nasal polyposis).

Other conditions that may be treated with a device as described herein include headache conditions, such as primary headaches (e.g., migraine, tension-type headache, cluster, trigeminal autonomic cephalgias, primary cough headache, primary exertional headache, primary headache associated with sexual activity, hypnic headache, new daily persistent headache, hemicrania continua, and primary thunderclap headache), secondary headaches (e.g., headache due to head and/or neck trauma, cranial or cervical vascular disorder, infection, disorder of homeostasis, headache or facial pain due to disorder of cranium, neck, eyes, ears, nose, sinuses, teeth, mouth, or other facial or cranial structures), and cranial neuralgias (e.g., trigeminal neuralgia, glossopharyngeal neuralgia).

CNS diseases that may be treated by administration of one or more therapeutic agents by a microneedle device according to the invention include CNS diseases caused by trauma, infections (e.g., microbial or viral), degenerative disorders (e.g., degenerative spinal or brain disorders), structural defects (e.g., birth defects, hypospadias), tumors, autoimmune disorders, and stroke. Examples of such CNS diseases include but are not limited to encephalitis, meningitis, tropical spastic parapesis, arachnoid cysts, Huntington's disease, locked-in syndrome, Parkinson's disease, Tourette's syndrome, multiple sclerosis, and Alzheimer's disease.

In embodiments where it is desirable for the administration of a compound to have a direct effect on the sinuses, the compound is typically administered to the middle meatus (including the osteomeatal complex) and/or to the superior meatus.

In certain embodiments, a compound is administered to the nasal and/or sinus mucosa by first coating the compound onto at least a portion of the one or more microneedles attached to a substrate of a device of the invention, and then inserting the end of the device with the coated microneedles into the nasal cavity, pressing the microneedles against the targeted nasal and/or sinus mucosal region such that at least a portion of the one or more microneedles penetrates the nasal and/or sinus mucosa, and then removing the device from the nasal cavity. A compound may be coated onto a microneedle by any suitable means, including, for example, by dipping the microneedle in a solution or gel containing the compound dissolved or suspended therein.

Optionally, the nasal cavity may be topically decongested and/or anesthetized prior to nasal administration of a compound with a device of the invention. For example, in certain embodiments, the nasal cavity may be topically decongested (e.g., with oxymetazoline) and/or anesthetized (e.g., with pontocaine) prior to nasal administration of a compound with a device of the invention.

In some embodiments, the device comprises an absorbent material (e.g., a MEROCEL® sponge) at one end of the device, and prior to application of the compound in the nasal and/or sinus mucosa, the region is first wiped down with the absorbent material. In other embodiments, the nasal and/or sinus mucosa is wiped down after application of the compound. In yet other embodiments, the mucosa is wiped down both before and after application of the compound.

The device of the invention provides one of ordinary skill in the art with an efficient, convenient, and safer means by which to nasally administer a composition, such as a therapeutic agent, to an individual. For example, in certain embodiments, one of ordinary skill in the art can wipe off a target area in the nose of a patient with an absorbent material at one end of the device, then flip the device around and administer a composition to the target area with composition-coated microneedles on a substrate at the other end of the device, all while continuing to observe the target area in the nose of the patient. As such, the artisan can focus on administering a composition to a target area in the nose of a patient without losing sight of the target area between wiping it down and administering the composition, for example, as may occur when using multiple instruments.

As discussed above, there are several characteristics of the sinonasal cavity that pose significant challenges to developing the ideal nasal delivery device. First, visibility is extremely limited, as one can routinely see only as far as the nasal vestibule and perhaps the anterior nasal septum. The region is also highly vascular and prone to significant bleeding. In addition, the mucosa of the nasal cavity is not uniform and has different absorptive qualities depending on the region. The nasal mucosa is also a highly sensitive region. Often, any manipulation leads to sneezing, pain and reflex discharge. Moreover, there are chemical barriers in the form of gels and enzymes that line the nasal cavity and interfere with absoption. These limitations can be readily overcome by a handheld device as described herein to provide painless and bloodless entry of medication to the nasal and sinonasal cavity.

There is a large unmet clinical need to provide a more applicable method of transnasal delivery for medications that treat nasal and non-nasal conditions. Microneedle technology has not yet been clinically adapted to the nose and/or sinuses because of the substantial limitations that the nose and sinus cavities pose to this technology. The nasal lining however, is actually well poised to provide a substantial surface to administer medication for nasal or sinonasal and non-sinonasal conditions because of its large surface area and highly vascular nature.

A nasal delivery device as described herein enables ready access to the nasal cavity in a safe and inexpensive way, thereby providing health care providers the ability to provide different therapies as well and thus increasing accessibility to different forms of treatment. Moreover, nasal administration of a composition with a device as described herein can be done on awake patients, and can be performed readily in the office (e.g., on an outpatient basis), with little or no preparation.

For sinonasal conditions, medication can be precisely delivered to problematic regions of the nasal cavity with a nasal delivery device as described herein. Delivery of medication only to problematic areas would lessen the total dose of medicine required to achieve a desired therapeutic effect. When compared with topical formulations, the therapeutic efficacy of the dose delivered by microneedles with a device of the invention would likely be higher, which would lessen the total dose of medication needed as well. Creating micropores in the superficial nasal mucosa would allow entry of molecules into the mucosal layer or superficial submucosal layer which may be devoid of blood vessels and nerve endings, thereby overcoming the shortfall of injection techniques.

The invention will now be further described by way of the following non-limiting examples.

### EXAMPLES

### Example 1

A 43 year old male has allergic rhinitis. Physician topically decongests the nasal cavity with oxymetazoline and anesthetizes it with pontocaine. A cotton-tipped applicator with a cotton tip comprising a microneedle array is dipped in methylprednisolone (vial contains total of 20 mg). Nasal speculum used to spread nostril. The opposite end of the applicator contains an absorbent material that is used to wipe off the nasal mucosa along the anterior and middle section of the inferior turbinate and septum. While keeping eyes on the region just wiped, the physician switches the cotton-tipped applicator around to insert the end with microneedles into the nasal cavity, which is then applied to turbinate and septal mucosa.

### Example 2

A 70 year old male has rhinitis and sinusitis. Physician topically decongests the nasal cavity with oxymetazoline and anesthetizes it with pontocaine. A cotton-tipped applicator with a cotton tip comprising a microneedle array is dipped in methylprednisolone (vial contains total of 20 mg). Nasal speculum used to spread nostril. The opposite end of the cotton-tipped applicator contains an absorbent material which is used to wipe off the nasal mucosa along the superior aspect of the inferior turbinate and middle turbinate. While keeping eyes on the region just wiped, the physician switches the cotton-tipped applicator around to insert the end with microneedles into the nasal cavity, which is then applied to the region of the middle meatus. The middle meatus is treated with medications and the patient's headache, drainage, pressure resolve.

### Example 3

A 36 year old female has recurrent sinusitis. Physician topically decongests the nasal cavity with oxymetazoline and anesthetizes it with pontocaine. A cotton-tipped applicator with a cotton tip comprising a microneedle array is dipped in methylprednisolone (vial contains total of 20 mg). Nasal speculum used to spread nostril. The opposite end of the cotton-tipped applicator contains an absorbent head which is used to wipe off the nasal mucosa along the superior aspect of the inferior turbinate and middle turbinate. While keeping eyes on the region just wiped, the physician switches the cotton-tipped applicator around to insert the end with microneedles into the nasal cavity, which is then applied to the region of the middle meatus. The middle meatus is treated with medications allowing it to become decongested and the episodes of sinusitis resolve.

### Example 4

A 40 year old male has allergic rhinitis. Unresponsive to traditional nasal steroid sprays because of compliance. Nasal cavity is topically decongested with oxymetazoline and anesthetized with pontocaine. Nasal speculum used to spread nostril. A cotton-tipped applicator with microneedles is dipped in premixed botulinum toxin type A (BOTOX®, DYSPORT®). The opposite end of the cotton-tipped applicator contains an absorbent material that is used to wipe off the nasal mucosa along the superior aspect of the inferior turbinate and middle turbinate. A total of 30 units of botulinum toxin type A is applied to turbinate mucosa, septal mucosa and middle meatus. The patient experiences relief of allergic symptoms.

### Example 5

A 25 year old female has non-allergic rhinitis. Unresponsive to traditional nasal steroid sprays because of compliance. Nasal speculum used to spread nostril. A cotton-tipped applicator with microneedles is dipped in premixed botulinum toxin type A (BOTOX®, DYSPORT®). The opposite end of the cotton-tipped applicator contains an absorbent material that is used to wipe off the nasal mucosa along the superior aspect of the inferior turbinate and middle turbinate. A total of 30 units of botulinum toxin type A is applied to turbinate mucosa, septal mucosa and middle meatus. The patient experiences relief of allergic symptoms.

### Example 6

A 52 year old male has allergic rhinitis. Unresponsive to traditional nasal steroid sprays because of compliance. Nasal speculum used to spread nostril. A cotton-tipped applicator with microneedles is dipped in premixed botulinum toxin type A (BOTOX®, DYSPORT®). The opposite end of the cotton-tipped applicator contains an absorbent head that is used to wipe off the nasal mucosa along the superior aspect of the inferior turbinate and middle turbinate. A total of 30 units of botulinum toxin type A is applied to turbinate mucosa, septal mucosa and middle meatus. The patient experiences relief of allergic symptoms.

### Example 7

A 47 year old female has non-allergic rhinitis. Unresponsive to traditional nasal steroid sprays because of compliance. Nasal cavity is topically decongested with oxymetazoline and anesthetized with pontocaine. Nasal speculum used to spread nostril. A cotton-tipped applicator with microneedles is dipped in premixed botulinum toxin type A (BOTOX®, DYSPORT®). The opposite end of the cotton-tipped applicator contains an absorbent material that is used to wipe off the nasal mucosa along the superior aspect of the inferior turbinate and middle turbinate. A total of 30 units of botulinum toxin type A is applied to turbinate

### Example 8

A 34 year old male has allergic rhinitis. Unresponsive to traditional nasal steroid sprays because of compliance. Nasal cavity is topically decongested with oxymetazoline and anesthetized with pontocaine. Nasal speculum used to spread nostril. A cotton-tipped applicator with microneedles is dipped in premixed botulinum toxin type B (MYOBLOC®) and total of 800 units of botulinum toxin type B is applied to turbinate mucosa, septal mucosa and middle meatus.

### Example 9

A 43 year old female has nonallergic rhinitis. Unresponsive to traditional nasal steroid sprays because of compliance. Nasal speculum used to spread nostril. A cotton-tipped applicator with microneedles is dipped in premixed botulinum toxin type B (MYOBLOC®) and total of 800 units of botulinum toxin type B is applied to turbinate mucosa, septal mucosa and middle meatus.

### Example 10

An 8 year old boy has nasal congestion and snoring because of enlarged turbinates. Physician topically decongests the nasal cavity with oxymetazoline and anesthetizes it with pontocaine. A cotton-tipped applicator with a cotton tip comprising a microneedle array is dipped in methylprednisolone (vial contains total of 10 mg). Nasal speculum used to spread nostril. The opposite end of the applicator contains an absorbent material that is used to wipe off the nasal mucosa along the anterior and middle section of the inferior turbinate and septum. While keeping eyes on the region just wiped, the physician switches the cotton-tipped applicator around to insert the end with microneedles into the nasal cavity, which is then applied to turbinate and septal mucosa.

### Example 11

A 60 year old male has tissue swelling after recent sinus surgery. It is determined that the turbinates or middle meatus has to be decongested. Physician topically decongests the nasal cavity with oxymetazoline and anesthetizes it with pontocaine. A cotton-tipped applicator with a cotton tip comprising a microneedle array is dipped in methylprednisolone (vial contains total of 20 mg). Nasal speculum used to spread nostril. The opposite end of the applicator contains an absorbent material that is used to wipe off the nasal mucosa. The steroid is then applied to the local area of swelling of the turbinates or middle meatus. While keeping eyes on the region just wiped, the physician switches the cotton-tipped applicator around to insert the end with microneedles into the nasal cavity, which is then applied to the turbinates or middle meatus.

### Example 12

A 34 year old male has allergic rhinitis. Unresponsive to traditional nasal steroid sprays because of compliance. Nasal cavity is topically decongested with oxymetazoline and anesthetized with pontocaine. Nasal speculum used to spread nostril. A plastic-tipped applicator with microneedles is dipped in premixed botulinum toxin type B (MYOBLOC®) and total of 800 units of botulinum toxin type B is applied to turbinate mucosa, septal mucosa and middle meatus.

### Example 13

A 43 year old female has nonallergic rhinitis. Unresponsive to traditional nasal steroid sprays because of compliance. Nasal speculum used to spread nostril. A plastic-tipped applicator with microneedles is dipped in premixed botulinum toxin type B (MYOBLOC®) and total of 800 units of botulinum toxin type B is applied to turbinate mucosa, septal mucosa and middle meatus.

### Example 14

A 47 year old female with severe pain in the lower half of the body due to cancer pain from vertebral bone metastases is successfully treated with transnasal application of betamethasone into the olfactory epithelium. Microneedle delivery with a microneedle device according to the invention is achieved using anterior rhinoscopy or under endoscopic control in the office, directly to the neuroepithelium. Microneedle application occurs once every 6 weeks and is associated with far fewer complications than spinal injections.

### Example 15

A 35 year old male with severe postherpetic neuralgia is treated with intrathecal administration of streroids to relieve his pain. It is discovered that transnasal application of steroids using a microneedle device is more effective and carries far fewer complications than traditional spinal injections. The dose of steroids is delivered using a microneedle device according to the invetion.

### Example 16

A 78 year old female with early Alzheimer's disease has dexamethasone delivered to the roof of the nasal cavity in the vicinity of the neuroepithelium to treat and prevent progression of Alzheimers disease. The steroid is delivered using a microneedle device according to the invention.

### Example 17

A 35 year old male with diskogenic pain is successfully treated with transnasal deliver of steroids into the roof of the nasal cavity in the region of the olfactory neuroepithelium. A microneedle device according to the invention is used to accurately deliver medication.

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A nasal delivery device comprising a substrate suitable for administration of a composition to a nasal or sinus mucosa, wherein the substrate comprises one or more microneedles.

2. (canceled)

3. The nasal delivery device of claim 1, wherein the substrate is selected from the group consisting of: cotton, a sponge, an inflatable balloon, a probe, a roller, and a polymeric silicone elastomer.

4. The nasal delivery device of claim 1, wherein the at least one or more microneedles are 150 micrometers or less in length.

5. The nasal delivery device of claim 1, wherein the nasal delivery device comprises a reservoir in fluid communication with the microneedle.

6. The nasal delivery device of claim 5, wherein the reservoir comprises an injection port.

7. The nasal delivery device of claim 5, wherein the device comprises a shaft.

8. (canceled)

9. (canceled)

10. The nasal delivery device of claim 6, wherein the injection port is suitable for attachment of a syringe.

11. The nasal delivery device of claim wherein the one or more microneedles are hollow.

12. The nasal delivery device of claim 1, wherein the one or more microneedles are porous.

13. The nasal delivery device of claim 1, wherein the substrate is cotton, wherein the cotton is attached to one end of a shaft, and the one or more microneedles are attached or integrated in the cotton.

14. The nasal delivery device of claim 1, wherein the substrate is non-absorbent, wherein the non-absorbent substrate is attached to one end of a shaft, and the one or more microneedles are attached or integrated in the non-absorbent substrate.

15. The nasal delivery device of claim 14, wherein the non-absorbent substrate is a roller.

16. The nasal delivery device of claim 15, wherein the roller is cylindrical and rotates freely relative to the shaft.

17. The nasal delivery device of claim 15, wherein the roller is partially cylindrical.

18. The nasal delivery device of claim 14, wherein the shaft comprises an injection port.

19. The nasal delivery device of claim 18, wherein the injection port is suitable for attachment of a syringe.

20. The nasal delivery device of claim 19, wherein the shaft comprises a hollow center tubing.

21. The nasal delivery device of claim 7 comprising an absorbent or non-absorbent substrate at one end of the shaft and an absorbent substrate at the opposite end of the shaft.

22. The nasal delivery device of claim 21, wherein the absorbent substrate is a sponge.

23. The nasal delivery device of claim 1, wherein the substrate is concaved, and the one or more microneedles are attached or integrated on one or both sides of the concave surface.

24. The nasal delivery device of claim 1, wherein the substrate is convex, and the one or more of microneedles are attached or integrated on one or both sides of the convex surface.

25. The nasal delivery device of claim 1, wherein the substrate is flat, and the one or more of microneedles are attached or integrated on one or both side of the flat surface.

26. (canceled)

27. The nasal delivery device of claim 5, wherein the substrate is cotton, wherein the cotton is attached to one end of a shaft, and the one or more microneedles are attached or integrated in the cotton, and wherein the shaft comprises a fenestrated central tubing in fluid communication with the reservoir and the one or more microneedles.

28. The nasal delivery device of claim 7, wherein the device comprises a shaft selected from the group consisting of: a bendable shaft, a calibrated shaft, and a shaft with a raised barrier.

29. (canceled)

30. (canceled)

31. The nasal delivery device of claim 28, wherein the shaft is a shaft with a raised barrier, wherein the raised barrier is situated at most 2.5 cm from the end of the device comprising the substrate comprising one or more microneedles.

32. (canceled)

33. The nasal delivery device of claim 22, wherein the absorbent substrate is a polyvinyl alcohol sponge.

34. The nasal delivery device of claim 1, wherein the substrate is a polyvinyl alcohol sponge, wherein the one or more microneedles are attached or integrated onto one or both sides of the sponge.

35. (canceled)

36. The nasal delivery device of claim 34, wherein the polyvinyl alcohol sponge comprises a soft pliable plastic sheet, wherein the microneedles are attached or integrated onto the sheet.

37. The nasal delivery device of claim 1, comprising one or more compositions for administration to the nasal mucosa.

38. The nasal delivery device of claim 37, comprising the one or more compositions at predetermined dosages.

39. The nasal delivery device of claim 37, wherein the composition is selected from the group consisting of: adreno corticosteroids, antibiotics, antimigraine drugs, antiviral drugs, cardiovascular drugs, central nervous system drugs, autonomic nervous system drugs, diagnostic drugs, histamine, antihistamines, narcotics, sex hormones, inorganic compounds, vitamins, peptides, polypeptides, and proteins.

40. (canceled)

41. The nasal delivery device of claim 37, wherein the composition is coated onto at least a portion of the one or more microneedles.

42. A method for the nasal administration of a composition, comprising:
(a) contacting a composition with a nasal delivery device of claim 1, wherein the nasal delivery device comprises a substrate suitable for administration of the composition to a nasal or sinus mucosal surface of a subject in need thereof, wherein the substrate comprises one or more microneedles, and wherein the composition coats at least a portion of the one or more microneedles of the device; and
(b) contacting the nasal delivery device of (a) with a nasal or sinus mucosal surface of the subject such that the one or microneedles penetrate at least a portion of the mucosal surface,
wherein the composition is nasally administered to the subject.

43.-65. (canceled)

66. The method of claim 42, further comprising cleaning the nasal or sinus mucosal surface with an absorbent substrate located on the shaft at the end opposite the substrate comprising the one or more microneedles prior to contacting the surface with the one or more microneedles.

67. The method of claim 42, further comprising cleaning the nasal or sinus mucosal surface with a non-absorbent substrate located on the shaft at the end opposite the substrate comprising the one or more microneedles prior to contacting the surface with the one or more microneedles.

68. The method of claim 42, further comprising cleaning the nasal or sinus mucosal surface with an absorbent substrate located on the shaft at the end opposite the substrate comprising the one or more microneedles after contacting the surface with the one or more microneedles.

69. The method of claim 42, further comprising cleaning the nasal or sinus mucosal surface with a non-absorbent substrate located on the shaft at the end opposite the substrate comprising the one or more microneedles after contacting the surface with the one or more microneedles.

70.-74. (canceled)

75. The method of claim 42, wherein the nasal or sinus mucosal surface mucosal surface is selected from the group consisting of: nasal vestibule, turbinates, septum, lateral wall, middle meatus, superior meatus, inferior meatus, olfactory mucosa and epithelium, squamous lining, pseudostratified columnar epithelium, and neuroepithelium.

77.-80. (canceled)

81. A method for the systemic delivery of a composition, comprising:
(a) contacting a composition with a nasal delivery device of claim 1, wherein the nasal delivery device comprises a substrate suitable for administration of the composition to a nasal or sinus mucosal surface of a subject in need thereof', wherein the substrate comprises one or more microneedles, and wherein the composition coats at least a portion of the one or more microneedles of the device; and
(b) contacting the nasal delivery device of (a) with a nasal or sinus mucosal surface of the subject, such that the one or microneedles penetrate at least a portion of the mucosal surface,
wherein the composition is systemically delivered to the subject.

82. (canceled)

83. (canceled)

84. A method for the CNS delivery of a composition, comprising:
(a) contacting a composition with a nasal delivery device of claim 1, wherein the nasal delivery device comprises a substrate suitable for administration of the composition to a nasal or sinus mucosal surface of a subject in need thereof, wherein the substrate comprises one or more microneedles, and wherein the composition coats at least a portion of the one or more microneedles of the device; and
(b) contacting the nasal delivery device of (a) with an olfactory mucosal surface of the subject, such that the one or microneedles penetrate at least a portion of the olfactory mucosal surface,
wherein the composition is delivered to the CNS of the subject.

85. (canceled)

86. A method for the nasal administration of a composition, comprising:
(a) applying a solution or gel comprising a composition to a nasal or sinus mucosal surface of a subject in need thereof;
(b) contacting the nasal or sinus mucosal surface of the subject with a nasal delivery device of claim 1, wherein the nasal delivery devices comprises a substrate, wherein the substrate is a roller and comprises one or more microneedles; and
(c) rolling the roller of the microneedle device such that the one or more microneedles penetrate at least a portion of the nasal or sinus mucosal surface of the subject,
wherein the composition is nasally administered to the subject.
